(19) United States
(12) Patent Application Publication
(10) Pub. No.: US 2024/0184364 A1
(43) Pub. Date: Jun. 6, 2024

(54) Determination of optimal stimuli for an interface based on evoked potentials

(71) Applicant: ORANGE, Issy-Les-Moulineaux (FR)

(72) Inventors: Rozenn Nicol, Chatillon Cedex (FR); Cyril Plapous, Chatillon Cedex (FR); Lenaig Gueho, Chatillon Cedex (FR)

(21) Appl. No.: 18/524,733

(22) Filed: Nov. 30, 2023

(30) Foreign Application Priority Data
Dec. 1, 2022 (FR) 2212653

(51) Int. Cl. G06F 3/01; G08C 17/02

(52) U.S. Cl. CPC G06F 3/015; G08C 17/02

(57) ABSTRACT

A method and apparatus for determining an interface signal suitable for at least one user, this interface being of the type operating by detecting an evoked potential in a physiological signal of the user in reaction to a transmission of an interface signal intended for the user. In particular, following the transmission of at least one interface signal, referred to as a current interface signal, at least the current interface signal is selected as the interface signal suitable for the user when the transmission of the current interface signal has caused a measured physiological signal of the user to have an intensity exceeding a threshold. The physiological signal results from a reaction of the user to a transmission of the current interface signal, including a carrier first frequency and a modulation second frequency.

### CROSS-REFERENCE TO RELATED APPLICATIONS

This application claims priority to and the benefit of French Patent Application FR2212653, filed Dec. 1, 2022, the content of which is incorporated herein by reference in its entirety.

### TECHNICAL FIELD

This disclosure relates to the field of detecting potentials evoked by direct neuronal interfaces (or BCI hereinafter, for “Brain Computer Interface”).

### PRIOR ART

As an example below, the evoked potentials can be auditory, or, alternatively, visual, tactile, etc.

An evoked potential is a signal that appears in electroencephalogram (hereinafter EEG) signals when a user is exposed to a stimulus. For example, in the SSAEP approach (for “Steady-State Auditory Evoked Potential”), the stimulus is auditory and consists of a sound having a carrier frequency and to which sinusoidal amplitude modulation is applied. Equipment such as a headset with EEG signal sensors makes it possible to measure EEG signals and find the modulation frequency in these signals.

An example of a stimulus used for an SSAEP application is a sine wave at a frequency audible to all (for example 1000 Hz) modulated by a second sine wave, for example, at 37 Hz. The sine wave at 1000 Hz is called the carrier and makes it possible to hear the modulating signal at 37 Hz. The latter constitutes the information of interest.

Indeed, it is possible to find stimulation frequencies between approximately 1 and 200 Hz in the auditory areas of the brain. However, at this frequency range of 1 to 200 Hz, these are low frequencies and can be difficult to reproduce and/or hear. This is why a carrier frequency is used as a vector, enabling the user to hear these frequencies more easily. A stimulus (sine wave modulating at 37 Hz) can then generate a measurable auditory evoked potential in the brain at the same frequency of 37 Hz (plus any harmonics). This frequency is measurable in the EEG signals of the person exposed to the stimulus. The 37 Hz modulation frequency can therefore be found in the user's EEG signals.

However, there is no consensus in the scientific community regarding the best stimuli, meaning the stimuli capable of triggering the most powerful evoked potentials, and therefore offering the best readability in EEG signals.

There are several different ways of constructing stimuli, with the recommended frequency ranges varying greatly from one study to another. Part of the explanation undoubtedly lies in the fact that EEG signals are highly variable from one person to another and vary widely according to many parameters (fatigue, context, concentration, etc.). Certain frequencies may therefore be more easily detectable in some people than others. It can also vary in the same person over time and according to certain conditions (emotional state, external disruptions, fatigue depending on the time of day, etc.).

### SUMMARY

This disclosure improves the situation.

To this end, it offers a solution for obtaining the best stimuli for each person and in a given context.

According to a first aspect, a method is proposed for determining at least one interface signal suitable for a user, this interface being of the type operating by detection of an evoked potential in a physiological signal of the user in reaction to a transmission of an interface signal intended for the user. The method comprises, following the transmission of at least one interface signal, referred to as the “current interface signal”:

selecting at least the current interface signal as an interface signal suitable for the user when the transmission of the current interface signal has caused an intensity of a measured physiological signal of the user that is greater than a threshold, the physiological signal resulting from a reaction of the user to a transmission of the current interface signal, comprising a carrier first frequency and a modulation second frequency.

The aforementioned physiological signal may for example be an electroencephalogram signal (or “EEG signal” hereinafter).

The aforementioned interface can be of the BCI type for “Brain Computer Interface”, consisting of detecting in the EEG signal collected from a user of this interface a signal referred to as the “evoked potential” because it has the same frequency as the aforementioned modulation second frequency of the stimulus signal presented to the user.

Such a method then makes it possible to select the optimal forms of the stimulus signals in order to maximize the intensity of the evoked potentials generated. Such a selection of the best stimulation signals for a BCI interface has the advantage of adapting the properties of the stimuli to different users and different conditions of use (emotional state, fatigue, external disruptions). An interface device using these stimuli can therefore be more robust because it is personalized for a given user and for his or her conditions of use. Indeed, such Brain Computer Interface (or “BCI”) devices, of a reactive type because the transmission of evoked potentials comes in response to stimuli, are based on analysis of brain activity (by detecting evoked potentials according to a method such as “Steady-State Auditory Evoked Potential” (or “SSAEP”)). It is therefore advantageous for the signals causing the strongest evoked potentials to be those used primarily by the interface device.

Thus, it will be understood that the method may comprise:
transmitting at least one interface signal, referred to as the “current interface signal”,
receiving the physiological signal resulting from the user's reaction to the transmission of the current interface signal,
selecting at least the current interface signal as a suitable interface signal for the user when the transmission of the current interface signal has caused a measured intensity of the user's physiological signal above a threshold, the current interface signal comprising a first, carrier, frequency and a second, modulation, frequency.

In one embodiment, the transmitted interface signal may result from a transformation of the current interface signal.

For example, the method may comprise:
transforming the current interface signal as long as no interface signal selection suitable for the user is (or cannot be) made.

In one embodiment, the method may then comprise:
measuring the user's physiological signal, in reaction to the transmission of the current interface signal,
transforming the current signal, and
repeating the measurement and transformation a plurality of times, in order to retain a plurality of interface signals suitable for the user and for which their respective transmissions caused an intensity of the user's physiological signal that exceeded a threshold.

In one embodiment, this threshold can be a function of an average of the intensities successively measured.

Thus, in such an embodiment, a statistical analysis of the stimulation signals in relation to the reaction they trigger in the user is proposed.

In one embodiment, the transformation of the current signal is carried out by modification of at least one frequency among the aforementioned first and second frequencies, and/or by modification of at least one amplitude associated with a frequency among the aforementioned first and second frequencies, relative to an amplitude associated with the other frequency among the first and second frequencies.

These modifications can be made in successive steps, for example until satisfactory frequency ranges are reached, for a given individual.

Alternatively, however, the transformation of the current signal is carried out by adding random noise to a time-frequency representation of the current signal.

In addition, an interface signal that can be selected may comprise a noisy first portion, followed in time by at least a second, non-noisy portion, called “pause time”. This type of signal can cause evoked potentials with a high signal-to-noise ratio.

In one embodiment, the interface signal suitable for the user is selected by implementation of inverse correlation filters.

The so-called “inverse correlation filter” method is a psychophysical method that is conventionally used to study cognitive processes, without prior knowledge of the impact of a particular stimulus on the cognitive system of an individual. Subjects are subjected to such stimuli and the responses of these subjects are observed by conducting a statistical analysis of the responses associated with each stimulus in order to infer properties of underlying processes. It is thus possible to optimize the stimuli on the basis of the collected responses.

For example, if the random transformation of an original signal distorts the modulation frequency of this signal by changing it for example from 35 Hz to 37 Hz, and this transformed signal causes an intense evoked potential, then this 37 Hz modulation frequency can be the frequency of a new generation of signals to be tested on the same individual. It will thus be understood that randomly added noise can improve the interface signals.

In an embodiment where the selected interface signals are sound signals, the method may further include storing the data of signals selected as suitable for the user (for example the first and second frequencies and the associated amplitudes) with a view to reproducing several of these signals via speakers, simultaneously.

In such an embodiment, the user can listen simultaneously to several audio signals at different modulation frequencies. For example, these signals can be played through respective speakers spaced apart from each other to allow the user to focus on one of these signals in particular. By focusing on one of these signals, the user's brain generates a periodic wave having the same frequency as the modulation frequency of the signal on which he or she is focused. An evoked potential is therefore collected for this signal. If this signal (which thus generates an evoked potential) is associated with a particular command (for example increasing the volume of a television set), then detection of the corresponding evoked potential will generate a corresponding command (such as increasing the sound volume of that television set). If the user had focused on another sound signal (of a different modulation frequency for example), another command would have been executed (for example “change TV channel”, or some other command).

A suitable carrier frequency for a sound signal from such a BCI interface is for example between 500 to 3500 Hz. The modulation frequency can be between 1 and 100 Hz, and more particularly between 20 and 80 Hz.

These are then starting ranges in which the frequencies of the initial interface signals which can be tested can be selected then transformed so as to modify, for example, their carrier frequency and/or their modulation frequency.

As for evaluating the intensity of the physiological signal of a user, the measurement of this physiological signal for a user, in reaction to a transmission of a current interface signal, may include searching for a frequency in the physiological signal corresponding to the modulation second frequency of the interface signal.

More particularly, the “intensity of the physiological signal” of the user can be determined by estimating a signal-to-noise ratio of the physiological signal measured on the user.

Thus, in one embodiment, the method may comprise:
retaining at least the interface signal suitable for the user, for which the emission caused a maximization, at the modulation frequency, of the signal-to-noise ratio of the physiological signal measured on the user.

According to another aspect, a computer program is proposed comprising instructions for implementing all or part of a method as defined herein when this program is executed by a processor. According to another aspect, a non-transitory computer readable storage medium is proposed, in which such a program is stored.

According to another aspect, a device is proposed comprising a processing circuit for implementing the above method.

### BRIEF DESCRIPTION OF DRAWINGS

Other features, details, and advantages will become apparent upon reading the detailed description below and upon analyzing the appended drawings, in which:

FIG. 1 shows an example application of the above method to a BCI type of interface according to one embodiment.

FIG. 2 shows an example of a signal generated with a carrier frequency fp and a modulation frequency fm (above), and an evoked potential signal detected in a user with a frequency identical to that of the modulation frequency fm (below).

FIG. 3 shows an example of a method within the meaning of the present description, according to one embodiment.

FIG. 4 shows an example of a device within the meaning of the present description, according to one embodiment.

### DESCRIPTION OF EMBODIMENTS

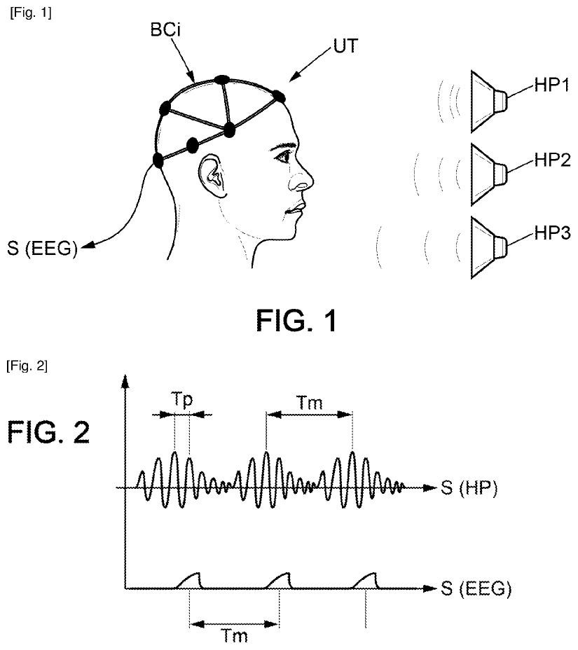

Reference is made first to FIG. 1 in order to remind of the principle of a BCI interface (“Brain Computer Interface”). A user UT wears a headset with electroencephalogram signal sensors (or “EEG signals” hereinafter), this headset possibly part of a general BCI interface. Different stimuli (sound, visual, or other) having respective modulation frequencies are delivered to the user, who focuses on one of the stimuli. The same modulation frequency as that of the stimulus on which the user is focused can then be measured in his or her EEG signals. In the example of a stimulus delivered by a light source, this source can flash at said modulation frequency. In the case of an audio signal, the signal may comprise sinusoidal modulation or may consist of successive beeps at said modulation frequency. An analysis of the user's EEG signals reveals the presence of a wave frequency corresponding to said modulation frequency. It is then possible to design a BCI interface in which several stimuli coming from respective sources HP1, HP2, HP3, having different modulation frequencies and representing for example different respective instructions for controlling a machine (for example “turn left”, “turn right”, “brake”), are presented simultaneously to a user and the user focuses on one of them such that his or her EEG signals reveal the frequency of one of the stimuli HPi and the function associated with this stimulus is then executed by the machine.

In the example in FIG. 1, the stimuli are delivered by respective speakers HP1, HP2, HP3 . . . and the user focuses on one of these sound sources. Thus, an embodiment is described below in which the interface signal (stimulus applied to user UT) is for example an audio signal in order to detect a physiological signal of the SSAEP type (for “Steady-State Auditory Evoked Potential”). The stimulus is therefore auditory and consists of a sound at a carrier frequency fp to which a sinusoidal or square wave modulation is applied, at a modulation frequency fm. The stimuli are generally constructed with a carrier frequency (corresponding to period Tp of FIG. 2), and modulated in amplitude, power, or energy, by a signal having a second frequency (corresponding to period Tm of FIG. 2). If the stimulation signal is effective, the EEG signal measured by the BCI interface headset has a frequency corresponding to period Tm in FIG. 2.

In the case of a visual stimulus, a light source can have a particular color, thus corresponding to a wavelength LO associated with a carrier frequency fp by a relation of the type LO=c/fp, where c is the speed of light. Some users may be more sensitive to certain colors than others in holding their attention. Thus, the choice of color (and therefore of the frequency of carrier fp) can be important, particularly at certain times of the day for the same user, in order to collect in an effective manner the EEG signals revealing an exploitable evoked potential (i.e. having a frequency corresponding to the modulation or “flashing” frequency of the light source). For example, a wavelength of 0.5 μm can be used for blue as the color of visual stimuli in certain subjects, or a wavelength of 0.55 μm for green to which some users are more sensitive, or 0.65 μm for red, a color to which other users are even more sensitive, etc.

It is thus understood that a subjectivity personal to each user means that certain stimuli are more easily perceived and can be the cause of evoked potentials that can be used to design a robust BCI interface for a given user.

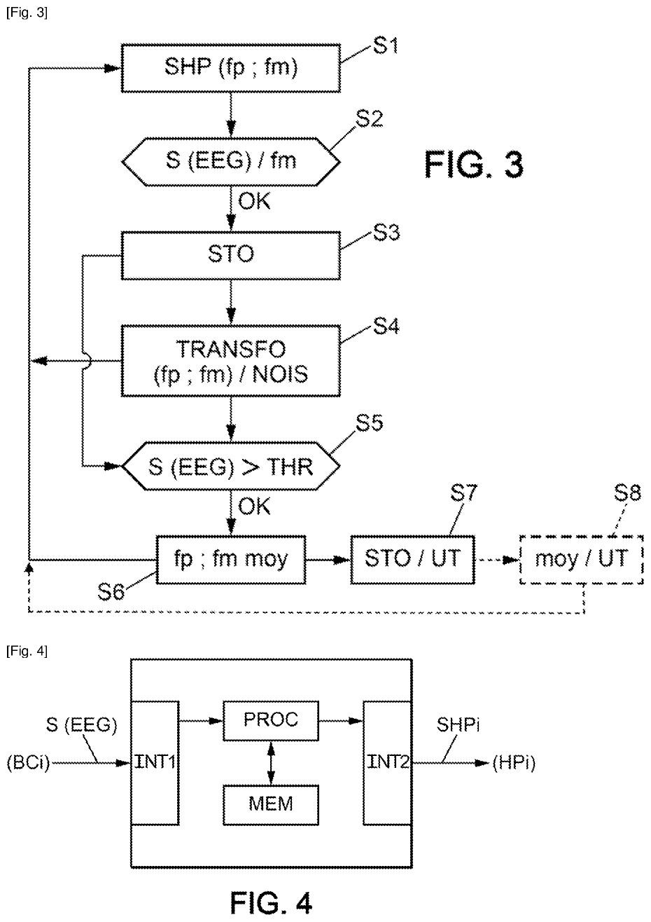

Presented below, with reference to FIG. 3, is a method for selecting the most effective stimuli for a given user or for an observed population of users. In step S1, a carrier frequency signal fp is generated with a frequency modulation fm, which is applied as a stimulus to a user. In step S2, the EEG signal is collected from the user and in particular it is sought to detect in this signal S(EEG) a frequency corresponding to modulation frequency fm. Stimulus signals SHP which have succeeded, for example, in generating an evoked potential in step S2 are stored in memory in step S3. From these stimulus signals, it can be sought, in step S4, to progressively transform successive versions of these signals:
by carrying out for example a modification by successive increments to modulation frequency fm and/or carrier frequency fp, and/or their respectively associated amplitudes in order to construct the stimulus signal SHP, according to a first embodiment, and/or
by applying random variations in noise, over time, to a time-frequency representation of the stimulus signals stored in memory in step S3, according to a second embodiment,
   then implementing an “inverse correlation filtering” technique, described in detail below.

Next, the transformations of stimuli having caused evoked potentials are in turn stored in memory (looping again over steps S1 to S3 of FIG. 1). In step S5, the transformed versions of the stimuli which caused evoked potentials with a signal-to-noise ratio (EEG signal at modulation frequency fm) greater than a threshold THR can then be retained. Threshold THR can be fixed, or on the contrary can be estimated based on the average of the signal-to-noise ratios. For example, all stimuli whose signal-to-noise ratio is greater than the average of the signal-to-noise ratios (possibly plus one or more standard deviations, for example) can be retained. In step S6, if there a few modulation frequencies fm and/or a few carrier frequencies fp appear for which the stimulus signals have provided particularly satisfactory results in test S5, stimulus signals can be constructed with frequencies fm and/or fp averaged among these “satisfactory frequencies” in order to test new versions of stimuli coming from these average frequencies (loop over steps S1 to S5). In particular, in the case of adding noise with random variation over time, it is possible to create a modulation by adding this noise which has the effect of randomly modifying the modulation frequency, or even the carrier frequency. In this case, the new versions tested can have these modified modulation and/or carrier frequencies. Finally, the best stimulus signals are stored in memory in step S7 (frequencies fm and fp, associated amplitudes, possibly a form of random noise modifying the signal, etc.). These “satisfactory” signals stored in step S7 can thus correspond to stimuli that are effective for generating evoked potentials.

It is also possible to store in step S7 signals having noisy portions, possibly with “pause times” between the noisy portions which correspond to the non-noisy signal.

In step S7, the tests carried out in steps S1 to S6 on a given user UT thus make it possible to obtain satisfactory stimulus signals for a BCI interface that this user would use. However, certain general trends have been observed, at least among populations of individuals, such that signals specific to a user UT could be more generally suitable for an entire category of users. Thus, in step S8, it is possible to average “satisfactory” signals according to tests carried out on several different users but for example belonging to the same category (such as an age group, a professional activity, or some other category). In this way, it is possible to further refine the individualization of stimulus signals specific to a user, once the user's category has been identified in step S8, by repeating the optimization steps for these signals (as illustrated by the dotted arrow from step S8 in FIG. 3). Thus, at the end of step S7, the signals obtained for a user can be used for the BCI interface of this user, and, in order to go further (in the dotted line embodiment of step S8 of FIG. 3), these signals tested on different users can be averaged to become standard signals from which individual optimizations can be pursued for each user (once their category has been identified).

For audio signals for example, it has been observed indeed that a range of carrier frequencies fp between 500 and 3500 Hz was promising for quickly obtaining evoked potentials for a large number of users. Modulation frequency fm is smaller in principle, for example within a range of 1 to 100 Hz. It has been observed in particular that between 20 and 80 Hz, evoked potentials can be detected for a large number of users, and younger users may be more sensitive to high frequencies (around 60 Hz up to 80 Hz).

The carrier signal can be modulated with this lower frequency fm, by a sine function or by a square wave. It is also possible to introduce silences into the carrier signal to generate a repetition in the form “signal—silence” at a frequency fm corresponding to that of said modulation. In step S4, it is also possible to modify the carrier signal amplitude just as the modulation amplitude can be modified.

Sound stimuli are thus successively generated with a modification of one of these parameters (frequency/amplitude; carrier/modulation; sine function/square wave) or several of these parameters. Evoked potentials that may possibly occur in the user's response to these stimuli are detected in the user's EEG signals. The aforementioned parameters of respective amplitudes and frequencies can be stored in memory in step S3, corresponding to a measurement of a signal-to-noise ratio of the EEG signals at this modulation frequency.

The manner in which said parameters are varied may be in progressive steps (for example incrementally increasing the modulation frequency from 20 Hz in order to identify a few modulation frequencies which are particularly suitable for the user because the evoked potentials detected present a high signal-to-noise ratio at these frequencies). Alternatively, the manner in which these parameters are varied may be random or may follow a predefined probability law.

In particular, one possible embodiment implements an “inverse correlation method”. This technique relies on the subjectivity of the tests. The transformation of the stimulus offered to the user is random and the user's reaction can be unexpected. For example, starting from a modulation frequency of 30 Hz, the user may react less well to 35 Hz than to 30 Hz, but better to 40 Hz than to 30 Hz, and does so in a reproducible manner. This can be explained by cognitive processes specific to the user.

Based on this observation, a time-frequency transformation is applied for example to the stimulus signals and a random noise (varying randomly over time) is added to this time-frequency representation in order to generate new stimuli to expose the user to. Such a transformation by adding random noise can modify, for example, the frequency of the carrier and/or of the modulation. If the user reacts to it with a high intensity of evoked potential (measured by its signal-to-noise ratio in test S5), then the signal characteristics of such a stimulus are stored in memory in step S7 to form part of the interface signals suitable for the user.

Thus, instead of fine-tuned variation of the identified parameters (frequency of the carrier, frequency of the modulating signal, amplitude of the carrier, amplitude of the modulating signal, etc.) as described in the first embodiment, in this second embodiment it is proposed to add noise to the stimuli in step S4 in order to create in step S6 a new set of stimuli to be evaluated (looping back to step S1 again).

Each subject UT listens to these signals. Synchronously, his or her EEG signals are recorded. It is then possible to determine which signals triggered the most powerful evoked potentials. The optimal stimuli can therefore be identified for a given user.

In one embodiment, it is then proposed to construct the best stimuli using the “inverse correlation” approach. The inverse correlation filter method is a psychophysical method for studying cognitive processes. For example, in other techniques, the image of a very noisy face can evoke different emotions in different subjects (for example fear for some individuals because the face is perceived to have an angry expression, or joy because the face is perceived by other individuals as smiling, for example). Inverse correlation thus uses the user's very personal response to a stimulus having added noise. The cognitive system is treated here as a black box. Subjects are exposed to these stimuli and their responses are observed. Statistical analysis of the responses associated with each stimulus makes it possible to deduce the properties of the underlying processes. It is thus possible to optimize the stimuli based on the responses collected. First, several stimuli are generated from a given stimulus. The given stimulus and the stimuli which constitute variants of the given stimulus form a set of stimuli to be tested. Then, at least some of the stimuli in this set of stimuli to be tested are successively reproduced.

Thus, here the audio stimuli are considered as an image by converting them into their time-frequency representation. The representation of their spectrogram can be obtained by dividing the signal into frames and transforming each frame into a frequency representation, to obtain a time-frequency image representing each stimulus.

Then, for each reproduced stimulus, an evoked potential is detected and a statistical analysis is carried out, in particular by inverse correlation of the detected evoked potential and the reproduced stimulus having triggered the detected evoked potential. In particular, for each evoked potential detected, the signal power (or the SNR as seen above) of the evoked potential is measured.

Stimuli are collected/selected from the reproduced stimuli on the basis of the result of the statistical analysis, in particular on the basis of the signal intensity of the detected evoked potentials (the stimuli corresponding, for example, to the detected evoked potentials whose signals are the most intense or to the detected evoked potentials whose intensity is greater than a given power threshold).

Optionally, the collected stimuli can be averaged, as described above. In particular, in order not only to optimize the stimuli but also to personalize them, the selection and/or averaging are carried out on the basis of detected evoked potentials for a given user.

In practice, several stimuli with different carriers and different modulation frequencies can be used. Each stimulus thus obtained is then transformed into an image using a time-frequency representation (spectrogram). Each spectrogram has the interference of random noise (the image being noisy). The resulting noisy spectrogram is then converted back into an audio signal.

These distorted audio stimuli can be used again, possibly a plurality of times, to feed a subjective test based on the principle of inverse correlation, as described above. Different subjects can listen to these signals. Their EEG signals are recorded synchronously. It is then possible to determine the signals that triggered the most powerful evoked potentials. “Most powerful” refers to the useful signal-to-noise ratio (SNR) of the EEG signal. It is sought to determine the stimuli maximizing the SNR in the frequencies measured in the EEG signals collected, in particular at the modulation frequency. For example, an SNR threshold can be defined above which the evoked potentials are considered to be robust and valid for detection. All stimuli meeting this criterion are collected. The waveforms of these stimuli can then be averaged individually for each user in order to obtain optimal personalized (i.e. individual) stimuli for a given user.

The above technique thus combines the following advantages:
Automatic selection of stimuli properties giving the best performance, particularly in terms of detectability and response time,
Automatic adaptation of a BCI interface to different users,
Robustness of detection in case of modification to conditions of use (emotional state, fatigue, external disruptions).

FIG. 4 illustrates one possible embodiment of a device for implementing the above technique. The device may typically include:
an interface INT2 for transmitting stimulation signals SHPi intended for example to be supplied to speakers (or light sources),
another interface INT1 for receiving EEG signals from the sensors of the BCI interface headset,
a processor PROC to carry out the tests of FIG. 3 in particular, and to identify the signals SHPi causing the most intense evoked potentials in the S(EEG) signals, and
a memory MEM storing the data of these signals SHPi in order to implement step S7 of FIG. 3 (or step S8 if averaging over several individuals is carried out).

In particular, memory MEM can be a block of memory which further stores instruction data of a computer program, the processor PROC cooperating with the memory MEM in order to read these data and execute these instructions.

The technique described above can find numerous applications using “reactive” type BCI interfaces (reaction to stimuli), for paralyzed or blind people, or even for “thought” control by means of a BCI interface headset (particularly in the context of controlling a smart home with commands such as “turn on the television”, “change the channel”, “turn on the light”, “turn off the heat”, etc.).

It should be noted that FIG. 4 may further represent the hardware elements of such a BCI interface. For example, the speakers HPi of the BCI interface can correspond to the earpieces of a headset in stereophonic playback and at least part of the signals retained in memory in step S7 can be played simultaneously by the earpieces of the headset in respective stereo “positions”.

1. A method for determining at least one interface signal suitable for a user, to be applied to an interface operating by detection of an evoked potential in a physiological signal of the user in response to a transmission of an interface signal intended for the user, the method being implemented by a device and comprising:
transmitting at least one interface signal referred to as a current interface signal; and
after transmitting the current interface signal, receiving a measured physiological signal of the user and selecting at least the current interface signal as an interface signal suitable for the user in response to the transmission of the current interface signal having caused the measured physiological signal of the user to have an intensity exceeding a threshold, the physiological signal resulting from a reaction of the user to the transmission of the current interface signal, the interface signal comprising a carrier first frequency and a modulation second frequency.

2. The method according to claim 1, wherein the transmitted interface signal results from a transformation of the current interface signal.

3. The method according to claim 1, comprising:
transforming the current interface signal as long as no interface signal suitable for the user has been selected.

4. The method according to claim 1, comprising:
measuring the user's physiological signal, in reaction to the transmission of the current interface signal,
transforming the current signal, and
repeating the measurement and transformation a plurality of times, in order to retain a plurality of interface signals suitable for the user and for which the respective transmissions caused the physiological signal of the user to have an intensity exceeding the threshold.

5. The method according to claim 4, wherein said threshold is a function of an average of the intensities successively measured.

6. The method according to claim 2, wherein the transformation of the current interface signal is carried out by modifying at least one frequency among the first and second frequencies.

7. The method according to claim 2, wherein the transformation of the current signal is carried out by modifying at least one amplitude associated with a frequency among the first and second frequencies, relative to an amplitude associated with the other frequency among the first and second frequencies.

8. The method according to claim 2, wherein the transformation of the current signal is carried out by adding random noise in a time-frequency representation of the current signal.

9. The method according to claim 1, wherein the selected signal comprises at least a first, noisy portion, followed in time by a second, non-noisy portion.

10. The method according to claim 1, wherein said at least one interface signal suitable for the user is selected by implementing inverse correlation filters.

11. The method according to claim 1, wherein the interface signals are sound signals, and the method further comprises data storage for the signals selected as being suitable for the user with a view to playback, by loudspeakers, of at least part of said selected signals, simultaneously.

12. The method according to claim 1, comprising measuring the physiological signal of the user, in reaction to the transmission of the current interface signal, by searching for a frequency in the physiological signal corresponding to the second frequency.

13. The method according to claim 1, wherein the intensity of the physiological signal of the user is determined by estimating a signal-to-noise ratio of the physiological signal measured in the user.

14. The method according to claim 12, wherein the intensity of the physiological signal of the user is determined by estimating a signal-to-noise ratio of the physiological signal measured in the user, the method further comprising:
retaining at least the interface signal suitable for the user, of which the transmission caused a maximization, at the modulation second frequency, of the signal-to-noise ratio of the physiological signal measured in the user.

15. A non-transitory computer-readable medium storing computer program instructions causing an implementation of the method according to claim 1, when the computer program is executed by a processing circuit of the device.

16. A device comprising:
a processing circuit configured to determine at least one interface signal suitable for a user, to be applied to an interface operating by detection of an evoked potential in a physiological signal of the user in response to a transmission of an interface signal intended for the user, the determining comprising:
transmitting at least one interface signal referred to as a current interface signal; and
after transmitting the current interface signal, receiving a measured physiological signal of the user and selecting at least the current interface signal as an interface signal suitable for the user in response to the transmission of the current interface signal having caused the measured physiological signal of the user to have an intensity exceeding a threshold, the physiological signal resulting from a reaction of the user to the transmission of the current interface signal, the interface signal comprising a carrier first frequency and a modulation second frequency.
